(19) United States
(12) Patent Application Publication
(10) Pub. No.: US 2017/0028009 A1
(43) Pub. Date: Feb. 2, 2017

(54) Methods and Compositions for Delivering an Extract of Piper Methysticum

(71) Applicant: Kambouris Shares Pty Ltd, Mildura Victoria (AU)

(72) Inventor: Ambrosios Kambouris, Mildura, Victoria (AU)

(73) Assignee: Kambouris Shares PTY Ltd., Victoria (AU)

(21) Appl. No.: 15/302,821

(22) PCT Filed: Apr. 3, 2015
(86) PCT No.: PCT/AU2015/050155

§ 371 Date: Oct. 7, 2016

Related U.S. Application Data
(60) Provisional application No. 61/977,240, filed on Apr. 9, 2014.

(51) Int. Cl. A61K 36/67; B65D 51/28; A61J 1/20; A61K 9/00; A61K 9/46

(52) U.S. Cl. CPC A61K 36/67; A61K 9/0053; A61K 9/0007; A61K 9/0095; A61K 9/0075; A61J 1/2093; A61J 1/2024; B65D 51/2821

(57) ABSTRACT

The present invention provides a method for delivering a P methysticum extract to an animal in need thereof, the method comprising the steps of: (i) administering the P methysticum extract orally to the animal, and (ii) administering an effervescent agent orally to the animal to cause the generation of an effervescence product within the alimentary tract of the animal, wherein the P methysticum extract and effervescence product co-locate at a site in the alimentary tract of the animal such that a psychoactive effect of the extract is improved relative to a method whereby there is no co-location of an effervescence product and the P methysticum extract. The method may be useful in altering the neurological state of a subject, to decrease anxiety, increase relaxation, decrease wakefulness, increase happiness, increase cheerfulness, or improve cognitive ability.

### FIELD OF THE INVENTION

The present invention is concerned with the pharmacological and recreational use of Piper methysticum extracts. In particular the invention relates to compositions (including beverages) comprising the root of P Methysticum.

### BACKGROUND TO THE INVENTION

P methysticum is grown as a crop and consumed generally in countries of the western Pacific including Vanuatu, Tonga, Samoa, and Fiji. Natives of these countries refer to the plant colloquially be a number of names including kava, kava-kava, 'awa, ava, yaqona and sakau.

The roots of P. Methysticum are used to produce a drink with sedative and anaesthetic properties. While kava drink is sedating, it is primarily consumed for relaxation, and is advantageous since it does not substantially affect other cognitive functions. The use of P methysticum for these purposes has become more widespread across first world populations in recent years. Kava root extract is now widely available in the world as a herbal supplement in the form of tablets, capsules, and dragees made of pharmaceutical grade extract. Commercially available kava formulations have been primarily ethanol or acetone extracts, standardized to a specified kavalactone content. The particular kavalactones in a Kava root extract vary depending upon its origin. The concentration ranges of total kavalactone levels in the Kava root extracts consumed are generally within the range of 10 to 30% by weight.

The psychoactive effects of P. methysticum are due to a heterogenous group of compounds known as kavalactones. The psychoactive ingredients of the Kava root have been identified as kavalactones. A number of kavalactone species have been identified, including kawain, dihydrokawain, methysticin, dihydromethysticin, yangonin, and desmethoxyyangonin. These compounds are substituted alpha-pyrones which are neutral, and nitrogen-poor. The lactone ring is substituted by a methoxy group in the C-4 position, and the compounds vary in their substitution by either a styryl residue (e.g., yangonin, desmethy-oxyyangonin, kawain, and methysticin) or by a phenylethyl residue (e.g., dihydrokawain and dihydromethysticin) at the C-6 position.

One disadvantage of the P methysticum extracts is that active kavalactones may be hydrophobic and therefore have poor solubility in water. (Hussain et al; Drug Dev. and Indust. Pharm. 23(12), 1223-1226; 1997). This causes difficulty in formulating a beverage, and also the absorption of actives into the serum.

The bioavailability of the various kavalactones vary (and vary independently) according to many factors. For example, the method of preparation. Traditionally prepared extracts are known to have different pharmacological properties to methods such as ethanol extraction. Other issues such as co-administration with food or alcohol may also affect things.

Another problem (which may be related to variable bioavailability) is the difficulty in achieving sufficient levels of kavalactones in the serum, or to reproducibly achieve sufficient levels for a desired effect. This may lead persons consuming excessive amounts of the extract, leading to potential hepatotoxicity in an effort to achieve a desired effect.

As extracts of P. methysticum root are obtained from a natural source, there are of course many compounds present other than kavalactones. Some may interfere with liver function, possibly leading to variations in the metabolism of kavalactones in the liver. Again, this may affect the ability to achieve a desired clinical effect.

It is a further problem that kava beverages may be unpleasant to consume. More traditionally prepared beverages are thought to have a lesser effect on the liver, however may be found to be unpalatable for some individuals.

It is a further problem that a desired neurological state may not be achieved, or may not be achieved to the required level using prior art kava preparations. For example, kava is consumed traditionally by Fijians to obtain the well known “cheerful” effect (Thompson et al; Hum Psychopharmacol. 2004 June; 19(4):243-50). This effect is highly desired, but cannot be obtained by the use of commercially available tablet-forms of kava. Even crushing tablets and dispersing into water for consumption does not provide the response. The response is obtainable by consuming traditionally prepared kava extracts in beverage form, however such extracts cannot be obtained commercially.

It is an aspect of the present invention to overcome or alleviate a problem of the prior art to provide methods and compositions for improving the pharmacokinetics (such as the bioavailability) of psychoactives in P Methysticum. It is a further aspect to provide a more palatable beverage of P Methysticum and/or an alternative beverage.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In one aspect, the present invention provides a method for delivering a P methysticum extract to an animal in need thereof, the method comprising the steps of
(i) administering the P methysticum extract orally to the animal, and
(ii) administering an effervescent agent orally to the animal to cause the generation
of an effervescence product within the alimentary tract of the animal,
   wherein the P methysticum extract and effervescence product co-locate at a site in the alimentary tract of the animal such that a psychoactive effect of the extract is improved relative to a method whereby there is no co-location of an effervescence product and the P methysticum extract.

In one embodiment, the effervescent agent consists of, or comprises, a chemical compound capable of releasing a gas upon exposure to moisture.

In one embodiment, the effervescent agent consists of, or comprises, an alkali metal carbonate salt, optionally in combination with an organic acid.

In one embodiment, the effervescence product is a gas

In one embodiment, the gas is carbon dioxide.

In one embodiment, the P methysticum extract is a dried preparation.

In one embodiment, the step of administering the extract occurs substantially contemporaneously with the step of administering the effervescent agent.

In a further aspect the present invention provides a dosage unit comprising or consisting of a dried preparation of P methysticum and an effervescent agent.

In a further aspect the present invention provides a method for altering the neurological state of a subject, the method comprising the steps of:
(i) administering the P methysticum extract orally to the animal, and
(ii) administering an effervescent agent orally to the animal to cause the generation of an effervescence product within the alimentary tract of the animal,
   wherein the P methysticum extract and effervescence product co-locate at a site in the alimentary tract of the animal.

In one embodiment of the method (i) the onset of alteration to neurological state is more rapid and/or (ii) the altered neurological state is of longer duration and/or (ii) the neurological effect is subjectively or objectively greater relative to a method whereby there is no co-location of an effervescence product and the P methysticum extract.

In one embodiment, the alteration of neurological state is a decrease in anxiety and/or an increase in relaxation and/or a decrease in wakefulness and/or an increase in happiness and/or cheerfulness, and/or improved cognitive ability.

In one embodiment, the alteration of neurological state is discernible by brain wave analysis, such as by EEG.

In one embodiment of the method the steps of:
(i) administering the P methysticum extract orally to the animal, and
(ii) administering an effervescent agent orally to the animal to cause the generation of an effervescence product within the alimentary tract of the animal,
   are achieved by the administration of the dosage unit as described herein.

Yet a further aspect of the invention provides a beverage comprising an effervescent agent, a P methysticum extract, and optionally an effervescence product.

In yet a further aspect the present invention provides a beverage container comprising
(i) a first region comprising a substantially aqueous liquid, and
(ii) a second region comprising a P Methysticum extract and an effervescent agent,
   wherein the container is configured such that the aqueous liquid contacts the P methysticum extract and effervescent agent in response to an action of a user of the container.

Further provided by the present invention in another aspect is a kit of parts comprising a P methysticum extract and an effervescent agent.

In one embodiment, the kit comprises instructions for the user to (i) contact the P methysticum extract and effervescent agent with an aqueous liquid, and to then (ii) ingest the liquid.

In one embodiment, the kit comprises a container.

### DETAILED DESCRIPTION OF THE INVENTION

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments covered by the claims.

Throughout the description and the claims of this specification the word “comprise” and variations of the word, such as “comprising” and “comprises” is not intended to exclude other additives, components, integers or steps.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

In a first aspect, the present invention provides a method for delivering a P methysticum extract to an animal in need thereof, the method comprising the steps of
(i) administering the P methysticum extract orally to the animal, and
(ii) administering an effervescent agent orally to the animal to cause the generation of an effervescence product within the alimentary tract of the animal,
   wherein the P methysticum extract and effervescence product co-locate at a site in the alimentary tract of the animal such that bioavailability and/or a pharmacological effect and/or palatability of the extract is improved relative to a method whereby there is no co-location of an effervescence product and the P methysticum extract.

Without wishing to be limited by theory in any way, Applicant proposes that effervescence positively affects the absorption of psychoactive compounds (such as kavalactones) from P methysticum extracts during passage through the gut. Thus, a subject in need of P Methysticum extract will be able to achieve a higher level, or a more consistent level of a psychoactive compound. This may allow for the use of smaller dosage units, and also lower the cost of production given the need for a lower amount of active per dose. It is further proposed that higher serums levels are achieved for a given dose of psychoactive compound.

Furthermore, and separate to the issue of bioavailability, the effervescence may ameliorate the effects of active compounds in P methysticum extract on the liver.

The effervescence product (such as CO₂), is preferably in the form of a dissolved gas at a partial pressure higher than would other normally be found in the lumen of the gastrointestinal tract. This effect may also be caused by any other effervescence products produced, such as a sodium acid salt (such as sodium citrate) that is produced in an effervescence reaction. As a conjugate base of a weak acid, sodium citrate may provide a buffering activity in the gastrointestinal tract thereby stabilizing pH and allowing for an environment more conducive to absorption of the actives.

The effervescence may also provide for a decrease the hydrophobicity of the P methysticum actives. An increase in water solubility generally translates into a higher rate of absorbance into the serum, or a higher amount of active partitioning into the extracellular fluid, thereby potentiating the pharmacological response of the subject.

A further advantage of the present method is that the effervescent agent may block histamine in the stomach to reduce the amount of hydrochloric produced. This may provide protection of any acid labile actives in the P methysticum extract.

The buffering effect of effervescent solutions increases pH of the stomach so that contents of the stomach may be more quickly passed to the small intestines. This may increase the rate of absorption of active compound into the serum.

The P methysticum extract, many be an extract from any part of the plant including a leaf, a root and a stem. Preferably, the extract is from the root (and particularly a rhizome of the root). The extract may be pressed from freshly picked kava root, or prepared from ground, shredded or powdered root. Alternative, extraction of actives with various organic and inorganic solvents such as an alcohol (typically ethanol), a fluorocarbon, or an acetate.

For administration, the extract may be mixed with a fatty solution such as full cream milk to improve the dissolution of the hydrophobic kavalactones. In some embodiments, this may be less important or unnecessary given the present of the effervescence product. The avoidance of fats may, for some individuals, improve the palatability.

The effervescent agent may be an elemental species, a combination of elementary species, a compound or a combination of compounds. The agent typically consists of, or comprises, a chemical compound capable of releasing a gas upon exposure to moisture. Again without wishing to be limited by theory it is proposed that the release of gas (typically in the form of bubbles) acts to improve the bioavailability or the total amount of active absorbed from the P methysticum extracts. For example, bubbling may facilitate the mixing of the P methysticum extracts with the contents of the gastrointestinal tract (such as semi-digested food). This mixing may shield the actives from direct contact with stomach acid to at least some extent, thereby limiting degradation of these compounds through the stomach.

Chemical compounds useful as an effervescent agent include alkali metal carbonate salts (such as sodium bicarbonate or sodium carbonate). Such salts are typically used in conjunction with, or formulated with a soluble, food grade organic acid such as citric acid, tartaric acid, malic acid, fumaric acid or adipic acid.

A typical effervescent reaction can involve citric acid and sodium bicarbonate as follows;

3NaHCO₃(aq)+H₃C₆H₅O₇(aq)->3H₂O(aq)+3CO₂(g)+Na₃C₆H₅O₇(aq)

This reaction suggests that 1 mole of sodium bicarbonate will produce 1 mole CO₂ gas. So if 252 mg sodium bicarbonate reacts completely, there will be 132 mg CO₂ gas, or 67.2 ml CO₂ gas (since STP; standard temperature and pressure, 1 mol of CO₂ is equal to 22.4 L).

From this equation, it can be seen that the amount of CO₂ produced is contingent on the amount of bicarbonate present in the presence of adequate amounts of citric acid and moisture.

Bicarbonate in typical effervescent mixtures can range from 5% to 45% by weight. The amount used would depends on the speed required for dissolution to occur. Speed of dissolution is also altered by the formulation into a powder (rapid), granules (slower) and tablets (slower again).

The effervescent agent may simply be a gas (such as carbon dioxide) which is in aqueous solution. The gas can be caused to bubble causing effervescence by, for example, by decreasing the pressure under which the solution is kept. For example, a sealed vessel may contain water having an amount of carbon dioxide dissolved therein. The sealed nature of the vessel maintains the gas in solution (at least to some extent). When the seal is broken (when the lid is removed, for example) the water effervesces due to release of pressure in the bottle. At that point, a dried preparation P methysticum may be added to the water upon which the effervescing water (along with the P methysticum extract) is consumed.

Given the benefit of the present specification, the skilled person appreciates that gases can be created in a liquid by various means alternative to those described above. All such means known to the skilled person are included in the ambit of this invention.

Given the benefit of the present specification, it will be apparent to the skilled person that many means exist to facilitate the co-localization off the effervescent agent and P methysticum extract at a target site in the gastrointestinal tract. For example, timing of the administration of the two components (more of which is discussed elsewhere herein), the use of tablet coverings to delay the release of the P methysticum extracts until a target site in the gastrointestinal tract is reached, means for delaying or facilitating gastric emptying, and like are all potentially useful.

In one embodiment, the P methysticum extract is administered as a dried preparation which releases psychoactive compounds upon exposure to moisture. Processes for the production of dried preparations of P methysticum include extraction with a volatile solvent, followed by evaporation of the solvent.

The prior art provides suitable dosage forms of P methysticum extract, many of which may be used in the context of the present methods. Powders, tablets, capsules, granules suspensions, and extracts are all known to the skilled person.

Dosages may be selected on the basis of weight of the subject, the variety of P methysticum from which the extract is obtained, the pharmacological effect required, the level of effervescent product and the like.

Dosage formulation may be adapted to providing for an acceptably short disintegration time while still providing for acceptable protection from stomach acid. Indeed, given the potentiating effect the effervescence reaction of the present invention, acid lability may be less of a consideration with the more important parameter being disintegration time.

Disintegration time may be especially important where the dosage unit is intended to be dissolved in water (or any other beverage) before ingestion. In such instances matrix forming excipients and high compression forces may well be avoided in formulation, with fine granules or even powder formulations being preferred.

The skilled person is familiar with methods of producing effervescent granules, powders and tablets. Given the advantage of using a dried preparation, dry methods such as slugging, direct compression and roller are preferred. The avoidance of avoiding methods requiring water may generally be avoided given the possible negative effect on cell viability and the possibility of reacting with the effervescent agent. However the use of organic solvents may avoid the problems by the use of aqueous liquids in formulation given their ability to evaporate comprehensively, and general inability to initiate an effervescence reaction.

It is to be appreciated that the co-localization can be achieved in circumstances whereby the effervescent agent is administered before, contemporaneously with, or after administration of the P methysticum extract.

As one example, the effervescent agent (in solid form) is administered firstly, followed by the administration of the P methysticum extract. In these circumstances, the delay between the two administrations cannot be so long that there is no co-localization of the effervescent agent and any psychoactive compound of the extract. Preferably, the delay in administration is no more than about 1 minute, but in some circumstances may be less than about 2, 3, 4,5, 6, 7, 8, 9 or 10 minutes.

As another example, the effervescent agent may be dissolved in a glass of water to commence the effervescence reaction, with the effervescing solution being ingested, and the reaction continuing in the gastrointestinal tract of the subject. Thus, the subject ingests an effervescent agent and an effervescence product (such as carbon dioxide), with the agent being converted to the product within the subject.

In a further example, the P methysticum extract in dried form is first administered, followed by the effervescent agent in solid form. In that circumstance, contact of both the agent and extract with moisture in the gastrointestinal tract will trigger the effervescence reaction, and also the release of active compounds. Water may be swallowed either before or after either or both administration steps to facilitate the effervescence reaction and release of active compounds.

In another example, a dried preparation of P methysticum extract and a effervescent agent are maintained separately until both are contact with water in a vessel. Upon the commencement of the effervescence reaction, the water is ingested by the subject. The effervescence reaction continues in the stomach, and the active compounds are released into the stomach.

In another example, the effervescent agent and P methysticum extracts are formulated into a dosage unit. The single dosage unit is either swallowed, or contacted with water in a vessel.

In one embodiment, the method results in an increase in bioavailability, rate of absorption or total amount absorbed by the subject of any one or more of kawain, dihydrokawain, methysticin, dihydromethysticin, yangonin, and desmethoxyyangonin. The increase in bioavailability, rate of absorption or total amount absorbed is at least 1%, 5%, 10%, 15%, 20%, 25%, 30%, 35%, 40%, 45%, 50%, 55%, 60%, 65%, 75%, 80%, 85%, 90%, 95%, or 100%, 110%, 120%, 130%, 140%, 150%, 160%, 170%, 180%, 190%, or 200% as compared with a method whereby no effervescence is co-located.

In one embodiment, the present method provides a subjective or objective neurological effect that has an onset that is at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% faster as compared with a method whereby no effervescence is co-located

In one embodiment, the present method provides a subjective or objective neurological effect that has a duration that is at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% longer as compared with a method whereby no effervescence is co-located.

In one embodiment, the present method provides a subjective or objective neurological effect that is at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% greater as compared with a method whereby no effervescence is co-located.

In another aspect, the present invention provides a dosage unit comprising a dried preparation of a P methysticum extract and an effervescent agent. This form of the invention provides a highly convenient means of delivering the P methysticum extracts to a target site in the gut. For example, the dosage unit may be dropped into a glass of water, effervesce and be consumed. Alternatively, the dosage unit may be swallowed, with effervescence occurring due to moisture in the stomach or water subsequently consumed.

The single dosage unit may be a capsule or a pellet comprising the P methysticum extract and an effervescent agent. Alternatively, it may be a sachet containing dried and powered P methysticum extract and a powdered effervescent agent.

Having the benefit of the present specification, the skilled person is capable of optimizing any aspect of the method, or any substance used in the method, or any dosage unit to optimize a pharmacokinetic parameter or positive effect of a P methysticum extract in the gastrointestinal tract. For example, the variety and amount of P methysticum extract may be the subject of routine experimentation

In optimizing the present methods to potentiate the pharmacological effect of P methysticum extract, the skilled person may implement existing methods for assessing serum levels of kavalactones, and also efficacy with respect to psychoactive effects.

In one embodiment, a dosage unit of the present invention may comprise at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, or 200 mg. Where the “cheerful” response is required, these dosages may be doubled, or even tripled.

Assays for kavalactones are known to the skilled person. For example, Shao et al (Journal of Chromatography A, Vol 825, Issue 1, 30 Oct. 1998, pp 1-8) disclose a reversed-phase high-performance liquid chromatographic method to simultaneously measure the six major kavalactones (methysticin, dihydromethysticin, kavain, dihydrokavain, demethoxyyangonin, and yangonin). The method uses the six compounds as external standards. The six compounds are completely separated within 35 min using a YMCbasic S-5 column and an isocratic methanol-acetonitrile-water-acetic acid (20:20:60:0.1, v/v) mobile phase at 40° C.

The skilled person is also familiar with studies useful in the assessment of the various claimed pharmacological effects of kavalactones. For example, assessment of the clinical efficacy of kavalactones in sleep disturbances associated with anxiety disorders is disclosed by Lehrl (Journal of Affective Disorders, Volume 78, Issue 2, February 2004, pp 101-110).

Assessment of pharmacokinetics of kavalactones are also known. Experimental work in the pharmacokinetics and disposition of the kavalactone kawain in vivo and in vitro is disclosed by Mathews et al (Drug Metab Dispos 2005, October; 33(10), pp1555-63).

The administration of the combination of P methysticum extract and effervescent agent may be by way of addition of same to any beverage, such as water, a dairy-based drink (including milk), a soft drink (or soda), mineral water, fruit juice, vegetable juice, alcoholic drink and the like. Accordingly, in a further aspect, the present invention provides a beverage comprising an effervescent agent, a P methysticum extract, and optionally an effervescence product. Where the beverage is already carbonated, the addition of the effervescent agent may enhance the pharmacokinetics of the P methysticum extract, or to potentiate a pharmacological effect.

In another aspect the present invention provides a beverage container comprising:
(i) a first region comprising a substantially aqueous liquid, and
(ii) a second region comprising a P methysticum extract and an effervescent agent,
   wherein the container is configured such that (a) the aqueous liquid contacts (b) the P methysticum extract and effervescent agent in response to an action of a user of the container.

A useful beverage container may comprise a blister pack (the second region) which is rupturable such that the contents (an effervescent agent and a P methysticum extract) are dropped into a vessel (the first region) disposed underneath and containing an aqueous liquid such as water.

In one embodiment the blister pack is characterized by a rupturable base and a cover of a formable material, the base and cover defining at least one pocket which accommodates a P methysticum extract and an effervescent agent. The blister pack is adapted be sealingly fitted over the neck portion of a container. In use, the vessel includes water and the user applies a force to the cover sufficient to rupture the rupturable base such that the P methysticum extract and effervescent agent are caused to drop into the container thereby contacting the aqueous liquid.

In one embodiment the opening is associated with a neck portion of said container, said base extending across the opening such that an outer perimeter thereof is fixed to the neck and acts as an anti-tamper seal.

In one embodiment the base ruptures when a minimum amount of force is applied on said cover, causing said element to fall into the container body.

In one embodiment the seal between the outer perimeter of the base and the container neck is such that rupture of said base to allow for said element to be added does not cause said seal to break.

In one embodiment the seal is such that the blister pack can be manually detached from the neck of the container.

In one embodiment the blister pack includes tabs which, when pulled, break said seal.

In one embodiment the container further includes a lid that is removably attachable from said neck such that when the lid is attached it envelops said blister pack.

In one embodiment the lid includes a means of sealing said opening when the blister pack has been removed.

In one embodiment the base is of a lower burst strength than said cover. In one embodiment the base is of aluminum.

In one embodiment the cover is of thermoformable or coldformable plastic.

In one embodiment the rupturable base is porous and adapted to diffuse oxygen through said blister pack.

A particularly suitable container is described in published international patent application WO 2012/045116, the contents of which is incorporated herein by reference in its entire.

In another aspect the present invention provides a kit of parts comprising a P methysticum extract and an effervescent agent.

In one embodiment, the kit provides comprises instructions for the user to (i) contact the P methysticum extract and effervescent agent with an aqueous liquid, and to then (ii) ingest the liquid.

In one embodiment, the kit provides instructions for the user to ingest the P methysticum extract and the effervescent agent. Preferably, the instructions are for the user to ingest the P methysticum extract and the effervescent agent contemporaneously.

The kit comprises a container, into which an aqueous liquid may be disposed, and into which a P methysticum extract and an effervescent agent may be added. The container may comprise instructions as described above.

### EXAMPLES

### Example 1

### Formulation of Dosade Unit (Unflavoured)

A dosage unit of the present invention was formulated as follows:

A dosage unit of the present invention was formulated by Ferngrove Pharmaceuticals (South Granville, NSW, Australia). Tables 1 and 2 show formulation details of a preferred dosage unit. The dosage unit contained 2.4 g of dried kava extract (having a standardised equivalent of 60 mg kavalactones). Sodium bicarbonate and citric acid were used as the effervescent agent (with carbon dioxide being generated, in use, as an effervescence product). Excipients including inulin, lactose and colloidal silica were added, to provide a 6.35 g dosage unit presented as a granular powder.

When added to water contained in a vessel, the dosage unit effervesced to provide a pleasant tasting beverage.

### Example 2

### Single Subject Clinical Study Showing Neurological Effects of Effervescent P methysticum Extract by EEG

The enhanced efficacy of P methysticum extract as an effervescent composition was demonstrated in a clinical study. The study was performed in a manner generally consistent with that described in Schulz et al, Phytomedicine, Vol. 5(6), pp. 449-458.

The subject was an adult in good physical and mental health. The subject was assessed by a clinical psychologist, and gave informed consent for participation.

The subject had an uneventful medical history and had no history of non-medical drug use. The study subject abstained from any medication, coffee, tea and alcohol to avoid interaction effects.

The formulation was as described supra, with the exception that the dosage of active was the standardised equivalent of 125 mg kavalactones.

Three separate studies were conducted under the supervision of a clinical psychologist, with a period of at least 5 days between studies.

The subject was connected to an EEG apparatus. The EEG signals were recorded with gold electrodes, which were attached to the scalp at positions O1, O2, P3 and P4 according to the 10-20 international system.

The subject was initially seated and allowed to rest to obtain a baseline EEG. Once a baseline reading was gained, the dosage unit was placed into a glass of water and the resulting solution consumed by the subject while effervescing.

### Discussion of Results:

Schultz et al., 1998, found kava increased the power band (mv) in both the theta and alpha band whilst decreasing the beta band relative to baseline measurements in the F2C2 and OzT6 (RT & RS) regions of the brain after 2 hours of taking a single dose.

In the present study the effects of kava (125 mg kavalactones), as a single dose (either as non-effervescent tablet form or in a granular effervescent dosage form), on brain wave activity (alpha, beta and theta), were monitored using EEG (my readings) according to the protocol described Schultz et al., 1998. Furthermore, the subject kept a note book and stop watch and documented the presence or absence, and depth of relaxation at 10 minute intervals.

Results expressed as a percentage change in my activity relative to baseline, showed that kava in either tablet form (125 mg standardized), increased the power of the theta band and alpha band in all areas of the brain tested. In contrast, the beta band decreased. This is consistent with the findings by Schultz et al., 1998.

In comparison, kavalactones in the effervescent dosage form, elicited an increase in the power of the theta and alpha band that was at least two-fold that of the kava in tablet dosage form relative to baseline. Beta band reduced far more significantly (almost two-fold) with the effervescent kava compared to tablet kava.

After 2 hours of taking kava, the subject reported feelings of relaxation after having taken the 125 mg kavalactones in the effervescent form. Compared to feelings of relaxation elicited after taking the non-effervescent form, the subject reported in all three experiments that the feelings were of more rapid onset, greater in effect, and of longer duration.

These findings indicate that a standardized kavalactones dose in effervescent dosage form is more effective than when taken in tablet form when compared after two hours of taking a single dose of 125 mg kavalactones, in eliciting relaxation, an increase in theta and alpha wave activity and a reduction in beta wave activity. The effect was more rapid, of greater effect, and of an extended duration.

A fourth experiment was conducted whereby the subject was given an effervescent composition devoid of kavalactones as a blinded control. The subject did not report any significant feelings of relaxation over the 5 hours post administrations.

### Example 3

### Multiple Subject Clinical Study Showing neorological Effects of Effervescent P methysticum Extract by EEG and Questionnaire

The physiological or psychological impact of kavalactones on a disorder or mood, can also be evaluated by questionnaire analysis of the subject before and after administering a potentially neurologically active agent. Electroencephalographic (EEG) analysis of total or selective brain wave activity may also be used and comparing it to either the subjects' baseline EEG, or to a normal distribution of normal EEG has long been established for analyzing the effects of kavalactones.

In the present study, a standardized 125 mg kavalactones dose in either tablet (non-effervescent) or effervescent form (as per Example 1), was given to a group of 12 subjects.

The subjects did not take prescription or other drugs and were asked not to consume stimulants i.e. coffee or tea etc. on the day of testing. After arrival, they were asked to remain seated for at least 15 minutes prior to commencing EEG and questionnaire studies.

Studies were carried out using specialized EEG equipment and four electrodes were attached to the scalp of patients, in areas known as O1, O2, P3 and P4, according to International 1020 Systems. Such electrodes were attached with specific conductive glue until conductivity measurements between scalp and electrodes were satisfactory according to equipment conductivity testing.

After a 3 minute baseline analysis of these scalp regions with eyes open, the subjects were given either 125 mg of kavalactones in tablet form with water (non-effervescent), or the same amount in effervescent formulation. The EEG measurements were then carried out after 10 minutes, 30 minutes, 1 hour, 2 hours and 3 hours post consumption of the standardized kavalactones preparation.

The subjects were also asked to record when and if they felt any type of an effect after consumption of the kavalactones dose form, particularly if it reduced their wakefullness. They were also asked to describe such an effect and score it between wide awake (1) and almost asleep (10).

The main brain waves examined were: theta (6 to 8.5 Hz); Delta (1.5 to 6 Hz); alpha (8.5 to 12.5 Hz); beta (12.5 to 30 Hz), using an EEG device. The brain wave scanning was carried over 3 minutes, producing a scan that was complex that intercalated all frequencies at the same time. A computer program (Neuroguide) was used to extract and separate the frequencies and determine the amplitude of each frequency that was measured in microvolts.

The interpretation of the raw EEG data, depended on where, how long and how many scalp sites were measured. The simplest approach for determining what effect kavalactones had on brain wave activity (frequency and amplitude), was to subtract the computer determined baseline frequencies and amplitudes from subsequent scans that were under the effect of the kavalactones. Often, there would be a positive or a negative result, depending if the drug stimulated that brain wave frequency, or reduced it respectively. This change was easier presented as percentage increase relative to baseline to use each subject's baseline as a control. For the purpose of further simplicity, brain wave frequencies for theta and alpha were combined together and compared to baseline. Whereas beta waves were not combined. Results were statistically evaluated using ANOVA and found statistically valid.

Briefly, the results showed that kavalactones standardized for 125 mg, in either tablet or effervescent dosage form, positively increased the quantity and amplitude of both theta and alpha waves. In contrast, beta waves were reduced or not affected in some instances (Table 1).

A direct comparison of combining theta and alpha activity effects when the dosage form of kavalactones was tablets or effervescent powder form, revealed several notable findings (Table 1). These were:
1. Effervescence elicited an increase in theta and alpha waves at around 10 minutes post consumption. In contrast, such an effect was not seen so early when taking tablet with kavalactones of the same activity.
2. The amplitude of theta and alpha activity combined was 1.5 times greater relative to baseline after 2 hours of consuming a similar dose of kavalactones in the effervescent dosage form relative to baseline. In contrast, the tablet format was only 25% higher than baseline activity.
3. Theta and alpha brain wave activity had returned to baseline in the tablet format after 3 hours post consumption. In contrast, effervescent kavalactones remained 25% higher at this time point.
4. Beta wave activity after 10 minutes had not reduced after taking kavalactones in either tablet or effervescent format.
5. Beta activity subsequently reduced by a maximum of 10% relative to baseline, with either tablet or effervescent format at 2 hours. At 3 hours, beta wave activity was no different to baseline for both dosage forms.

Questionnaire analysis of subjects during the time course study revealed the following data summary:
1. 7 out of the 12 subjects felt that they were becoming relaxed at about 5 to 15 minutes post effervescent consumption. Their relaxation scores were between 4/10 and 6/10, translating to a reduction of feeling wide awake and becoming relaxed/sleepy. These subjects then rated their relaxation scores at between 7 and 10/10 after 2 hours and between 6 and 8 at 3 hours.
2. Another 3 out of the 12 subjects felt sleepy only after 30 minutes of taking the effervescent kavalactones dosage. These subjects then rated their relaxation scores at between 6 and 8/10 after 2 hours and between 5 and 6/10 at 3 hours.
3. The other 2 of the 12 subjects, felt relaxation of about 5 out of 10, after 2 hours and 2 to 4/10 at 3 hours.
4. 9 out of the 12 subjects taking tablets of standardized kavalactones, first noticed relaxation of between 3 and 5/10, after 1 hour post consumption of tablets. At 2 hours, this relaxation remained between 3 and 7 out of 10. By 3 hours, relaxation was between 2 and 3/10.
5. 3 out of 12 subjects did not see relaxation greater than 1 to 3 at 2 and 3 hours after taking the tablet dose.

TABLE 1
EEG measurement of brain wave activity and relative
changes as a result of standardized 125 mg Kavalactones
dosage as either tablets or effervescent form.

| Dosage Form | | | 10 | | | |
|---|---|---|---|---|---|---|
| kavalactones | Wave | | minutes | 1 hour | 2 hour | 3 hour |
| 125 mg | form | | post | post | post | post |
| standardized | monitored | baseline | admin | admin | admin | admin |
| Tablet | Theta & | 1 | 1 | 1.1 | 1.25 | 1.03 |
| formulation | Alpha | | | | | |
| (Non- | | | | | | |
| effervescent) | | | | | | |
| | Beta | 1 | 1 | −1.1 | −1.1 | 1 |
| Example 1 | Theta & | 1 | 1.1 | 1.2 | 1.5 | 1.25 |
| formulation | Alpha | | | | | |
| (Effervescent) | | | | | | |
| | Beta | 1 | 1 | −1.1 | −1.1 | 1 |

### Example 4

### Clinical Study Showing Elicitation of “Cheerful” Response by Administration of Effervescent P methysticum

The so-called “cheerful” response is a well known and highly desired neurological state elicited by the administration of kava. The response is distinct from the calming, relaxing and sedative effects of kava and is obtained especially by traditional Fijian tribal use of the extract. The cheerful response was known at the priority date to be not well reproduced by taking kava extract in prior art tablet form, hence the need to utilise the traditional Fijian kava extract preparations.

Applicant has surprisingly shown that the cheerful response may be obtained through the administration of an effervescent kava beverage.

A trial of 12 subjects already familiar with the cheerful response proceeded as follows.

6 subjects were randomly selected from the pool of 12. Each selected subject was orally administered 3 effervescent dosage units of Example 1 in water to provide a total of 375 mg kavalactones. The remainder 6 subjects were administered 375 mg kavalactones in tablet form (i.e. non-effervescent form).

All subjects receiving the effervescent dosage units reported the unequivocal achievement of a cheerful response. No subject receiving the effervescent dosage units reported the achievement of a cheerful response.

### Example 5

### Wildberry Flavoured Effervescing P methysticum Beverage

It is known that the taste of kava-based compositions are not agreeable to all consumers. The present invention therefore provides flavoured beverages, prepared as follows:

| Ingredient | Amount (g) | w/w % |
|---|---|---|
| Base (Example 1 | 46.64 | 93.28 |
| composition) | | |
| Flavour MTP-0056107 | 2.45 | 4.90 |
| Wildberry | | |
| Sucrolose | 0.66 | 1.32 |
| Citric Acid | 0.25 | 0.50 |
| TOTAL | 50.00 | 100.00 |

4 grams of the above composition was mixed in 250 ml water to provide a pleasant tasting beverage.

### Example 6

### Pina Colada Flavoured Effervescing P methysticum Beverage

It is known that kava-based compositions are not agreeable to all consumers. The present invention therefore provides flavoured beverages, prepared as follows:

| Ingredient | Amount (g) | w/w % |
|---|---|---|
| Base (Example 1 | 46.64 | 93.28 |
| composition) | | |
| Flavour MTP-005022 | 2.45 | 4.90 |
| Pineapple | | |
| Flavour MTP-010276 | | |
| Coconut | | |
| Sucrolose | 0.66 | 1.32 |
| Citric Acid | 0.25 | 0.50 |
| TOTAL | 50.00 | 100.00 |

4 grams of the above composition was mixed in 250 ml water to provide a pleasant tasting beverage.

### Example 7

### Beverage Container for Consuming Effervescing P methysticum Beverage

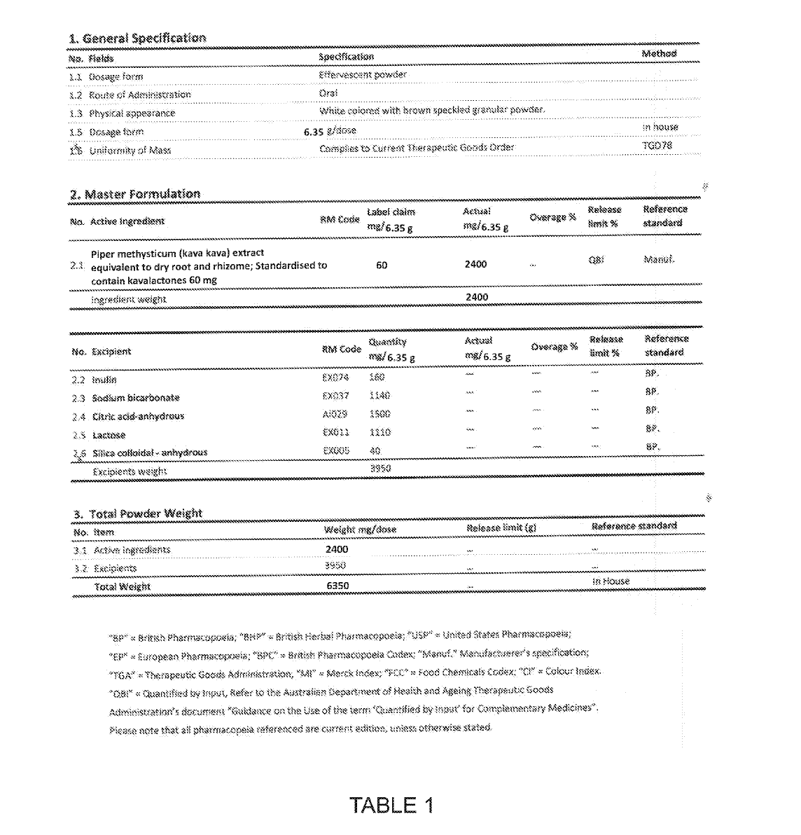

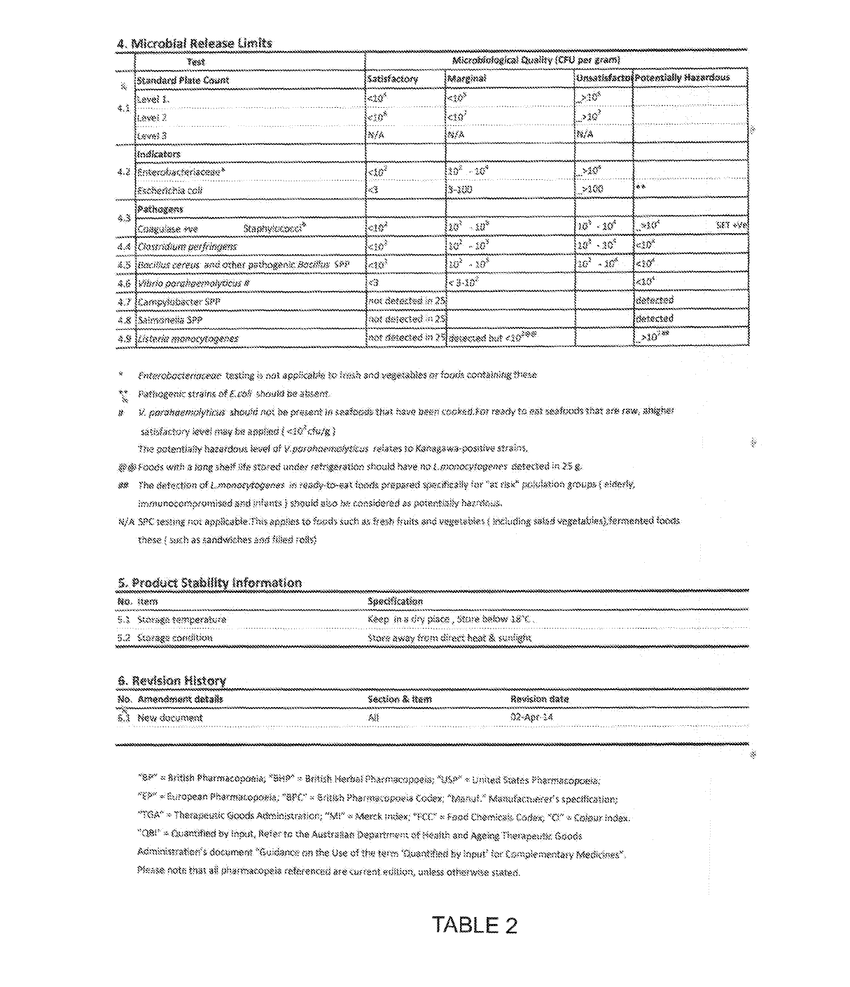

Reference is now made to FIGS. 1 to 5 which show diagrammatically a beverage container of the present invention 10 adapted to deposit a tablet comprising P methysticum extracts and an effervescent agent into a beverage, whereby the user unscrews the lid 12 from the beverage container 10, which may have a typical tamper evident sealing ring on the lid (not shown). With the lid 12 removed, the user is presented with the blister pack 14, which contains a dried P methysticum extract preparation, sodium bicarbonate and tartaric acid all co-formulated into a readily dissolvable tablet 16. The blister pack has instructions printed on the cover 18 instructing the consumer to press the tablet 16 into the beverage 28.

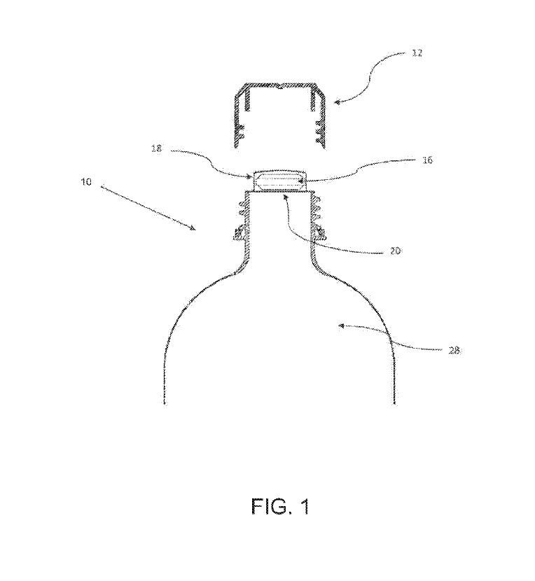

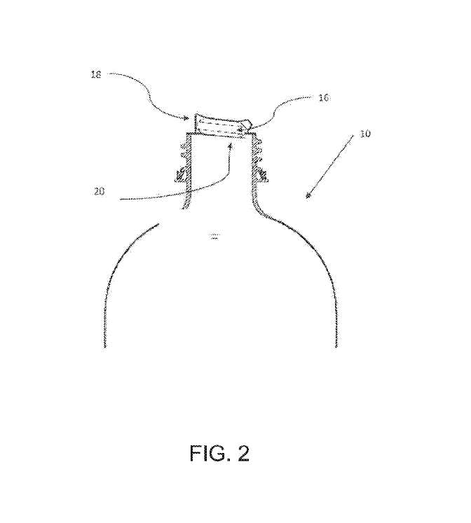

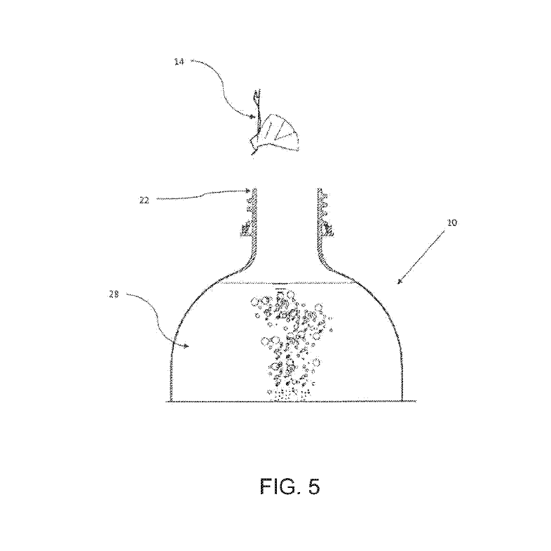

When force is exerted onto the cover 18, preferably by a thumb or some other manual means, it pushes against the tablet 16, forcing the rupturable base 20 to break as illustrated in FIG. 2. Once sufficient force is exerted to push the table 16 completely through the base 20, the tablet 16 falls into the container 10 as illustrated in FIG. 5.

The cover 18 is therefore indented into the neck of the container, however because of the durability of the material and the seal to the container neck 22 the cover 18 does not rupture or come away from the container 10, but remains sealed. This allows the user to twist or shake the container 10 to mix the beverage 28 with the tablet 16 without having to be cautious that the liquid may spill and allows the tablet 16 to be at least partially dissolved into the beverage 28 before consumption.

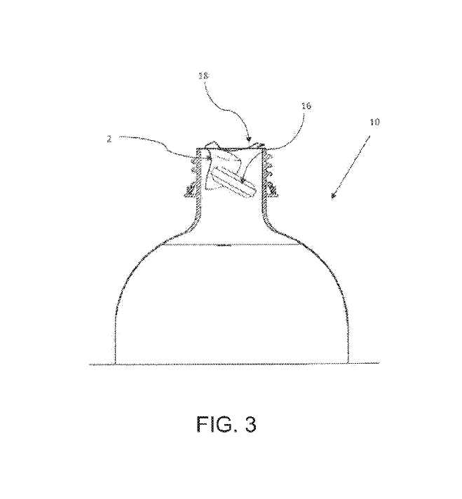

FIG. 3 shows rupture of the rupturable base 20, allowing the tablet 16 to enter the container.

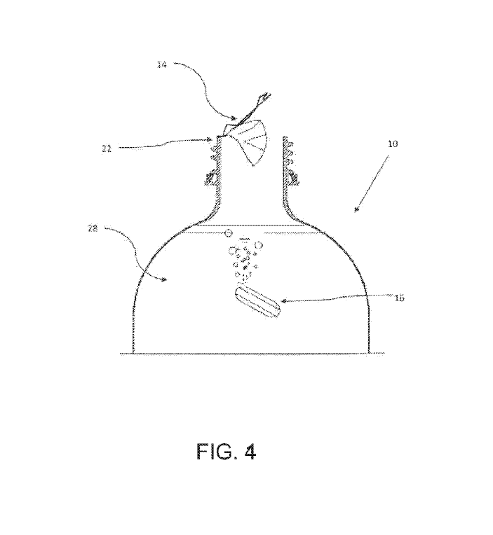

FIG. 4 shows the tablet 16 dissolving in the beverage. The bicarbonate and tartaric acid commence an effervescence reaction with the beverage to release viable P methysticum extracts into the beverage.

When the user is ready to drink the beverage, the rupturable base is completely removed from the container (FIG. 5).

It will be appreciated that in the description of exemplary embodiments of the invention herein, various features of the invention are sometimes grouped together in a single embodiment, figure, or description thereof, for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention.

1. A method for delivering a P methysticum extract to an animal in need thereof, the method comprising acts of
(i) administering the P methysticum extract orally to the animal, and
(ii) administering an effervescent agent orally to the animal to cause the generation of an effervescence product within the alimentary tract of the animal,
wherein the P methysticum extract and effervescence product co-locate at a site in the alimentary tract of the animal such that a psychoactive effect of the extract is improved relative to a method whereby there is no co-location of an effervescence product and the P methysticum extract.

2. The method of claim 1 wherein the effervescent agent consists of, or comprises, a chemical compound capable of releasing a gas upon exposure to moisture.

3. The method of claim 1 wherein the effervescent agent consists of, or comprises, an alkali metal carbonate salt, optionally in combination with an organic acid.

4. The method of claim 1 wherein the effervescence product is a gas

5. The method of claim 2 wherein the gas is carbon dioxide.

6. The method of claim 1 wherein the P methysticum extract is a dried preparation.

7. The method of claim 1 wherein the act of administering the extract occurs substantially contemporaneously with the act of administering the effervescent agent.

8. (canceled)

9. A method for altering the neurological state of a subject, the method comprising acts of:
(i) administering the P methysticum extract orally to the animal, and
(ii) administering an effervescent agent orally to the animal to cause the generation of an effervescence product within the alimentary tract of the animal,
wherein the P methysticum extract and effervescence product co-locate at a site in the alimentary tract of the animal.

10. The method of claim 9 wherein (i) the onset of alteration to neurological state is more rapid and/or (ii) the altered neurological state is of longer duration and/or (ii) the neurological effect is subjectively or objectively greater relative to a method whereby there is no co-location of an effervescence product and the P methysticum extract.

11. The method of claim 9 wherein the alteration of neurological state is a decrease in anxiety and/or an increase in relaxation and/or a decrease in wakefulness and/or an increase in happiness and/or an increase in cheerfulness, and/or improved cognitive ability.

12. The method of claim 9 wherein the alteration of neurological state is discernible by brain wave analysis.

13. The method of claim 12 wherein the brainwave analysis is EEG.

14. The method of claim 9 wherein the acts of:
(i) administering the P methysticum extract orally to the animal, and
(ii) administering an effervescent agent orally to the animal to cause the generation of an effervescence product within the alimentary tract of the animal,
are achieved by administration of a dosage unit a dosage unit comprising or consisting of a dried preparation of the P methysticum extract and the effervescent agent.

15. (canceled)

16. A beverage container comprising
(i) a first region comprising a substantially aqueous liquid, and
(ii) a second region comprising a P Methysticum extract and an effervescent agent,
wherein the container is configured such that the aqueous liquid contacts the P Methysticum extract and effervescent agent in response to an action of a user of the container.

17. (canceled)

18. (canceled)

19. (canceled)
